(19) United States
(12) Patent Application Publication
(10) Pub. No.: US 2015/0038454 A1
(43) Pub. Date: Feb. 5, 2015

(54) ANTIMICROBIAL COMPOSITIONS AND METHODS OF USE

(71) Applicant: Mary Malast, Chesterfield, MO (US)

(72) Inventor: Mary Malast, Chesterfield, MO (US)

(21) Appl. No.: 14/448,452

(22) Filed: Jul. 31, 2014

Related U.S. Application Data
(60) Provisional application No. 61/860,278, filed on Jul. 31, 2013.

(51) Int. Cl. A61K 31/7016; A61K 47/02; A61M 11/00; A61K 47/10; A61J 1/14; A61C 19/06; A61K 9/00; A61K 47/18

(52) U.S. Cl. CPC A61K 31/7016; A61K 9/0053; A61K 47/02; A61K 47/186; A61K 47/10; A61J 1/14; A61C 19/063; A61M 11/00
USPC 514/53; 222/420; 206/570; 604/93.01

(57) ABSTRACT

Compositions comprising an antimicrobial effective concentration of sucralose and methods of using for treating pharyngeal and other bacterial conditions.

### CROSS REFERENCE TO A RELATED APPLICATION

This application claims priority from U.S. Provisional Application Ser. No. 61/860,278 filed on Jul. 31, 2013 the disclosure of which is hereby incorporated by reference in its entirety.

### FIELD OF THE INVENTION

The present disclosure relates to antimicrobial compositions and methods of using the compositions for treating microbial conditions, and as such pharyngeal conditions.

### BACKGROUND OF THE INVENTION

The human mouth has an extraordinarily diverse microbiome, which includes viruses, archaea, protists, fungi, and bacteria. Most studies of the mouth microbiome have focused on bacteria and relationship of the bacteria to various disease pathologies. Varying approaches including newer sequencing technologies have shown that the individual human oral microbiome consists of at least about 700 and as many as 1200 bacterial species, 35% of which are not culturable in a laboratory setting. Bacterial diversity in the mouth is clearly extensive and the oral environment highly complex.

Dermal and pharyngeal bacterial pathogens are resident bacteria that cause a wide range of debilitating conditions when they adhere to pharyngeal surfaces. Conditions include tissue inflammation and swelling, pain, loss of appetite, tissue degeneration, tooth loss, halitosis, hearing loss, poor sense of smell and quality of life, loss of taste, and fatigue. Oral conditions such as periodontal disease and caries, especially, remain an important health issue despite over a hundred years of active research by dentists, physicians, and scientists. The control of biofilm accumulation on teeth has been the cornerstone of periodontal disease prevention for decades. Current treatment options include physical disruption of adhered bacteria and chemotherapeutic agents such as antibiotics or chlorhexidine, and are not sufficiently effective as evidenced by the widespread prevalence of gingivitis. Additionally, inflamed tissues and compound contours of the affected area make delivery of topical treatments problematic, especially where swelling results in complete cavity aperture or pathway occlusal. There is accordingly a need in the art for safe optimal compositions and methods for treating conditions caused by pharyngeal bacteria.

### SUMMARY OF THE INVENTION

In one aspect, the present disclosure provides a method of treating a pharyngeal condition in a subject, the method comprising delivering to a pharyngeal surface tissue in the oral cavity of the subject a composition comprising an antimicrobial effective concentration of sucralose, wherein the antimicrobial effective concentration of sucralose is at least about 1% w/w. In the method, the composition can be and preferably is sucrose-free. The oral cavity of the subject can also be substantially sucrose-free or sucrose-free when the composition is delivered. The composition can be delivered for example at least once daily over a period of at least about 1 week, 2 weeks or 3 weeks, or over a period of at least about 1 month. The method may further comprise maintaining the oral cavity substantially sucrose-free during a post-administration period immediately following delivery of the composition to a pharyngeal surface tissue. The post-administration period can be for example at least about 5 hours, or at least about 6-8 hours, or more, for example at least about 10-12 hours. The post-administration period can be for example during a regular daily quiescent period, for example during a daily period of sleep. Maintaining the post-administration period substantially sucrose-free can comprise, for example, avoiding food and drink during the post-administration period. Delivering to a pharyngeal surface tissue in the oral cavity of the subject can comprise contacting the pharyngeal surface tissue with the composition, for example a dropper, spray or other liquid applicator device. A pharyngeal condition can be selected from the group of swelling and inflammation of pharyngeal tissue, otitis media, gingivitis, periodontal disease, dental caries, halitosis, dermatitis, sinusitis including fungal sinusitis, pharyngitis, chronic rhinitis, tonsillitis, cough and chest congestion. For example, the pharyngeal surface tissue can be nasopharyngeal surface tissue and the nasopharyngeal surface tissue can be contacted with the composition using a nasal spray formulation. A nasal spray formulation can be administered 3 to 5 times daily, for example using 2 puffs per nostril per administration. Alternatively, the pharyngeal surface tissue can be oropharyngeal surface tissue and the composition is administered topically using a oral drop formulation such as a dental drop formulation. An oral drop formulation can be administered for example during and/or after tooth brushing. For example, 2 to 3 drops of an oral or dental drop formulation can be administered during and/or after tooth brushing.

In another aspect, the present disclosure provides an antimicrobial composition, wherein the composition comprises an antimicrobial effective concentration of sucralose, wherein the antimicrobial effective concentration of sucralose is at least about 1% w/w or more. The composition can be substantially sucrose-free. The composition can be for example a sucralose solution comprising about 1% to about 25% w/w sucralose. The sucralose solution can be prepared as a buffered saline solution. For example, a buffered saline solution can comprise about 0.35-0.45% w/w sodium chloride and about 0.1% to about 0.2% w/w hydrogen carbonate. A sucralose solution can further comprise a preservative, such as but not limited to benzalkonium chloride. A composition can be formulated for topical administration as a nasal spray, or as oral or dental drops, or any foaming or liquid oral composition such as a wash, lavage, coating or varnish. An antimicrobial composition may further comprise at least one additive such as a desensitizing agent, a flavoring agent, a coloring, a fluoride source, an emulsifier, a remineralizing agent, an antimicrobial agent, an anticalculus agent, an antioxidant, a vitamin, a teeth whitening agent, or combination of any thereof. For example, an antimicrobial composition may further comprise comprising hydroxylapatite and/or calcium phosphate as a remineralizing agent.

In another aspect, the present disclosure provides a kit comprising at least one container containing any antimicrobial composition described herein, or multiple containers containing components for preparing any antimicrobial composition described herein. Any kit may further contain at least one delivery or applicator device for delivering or administering the antimicrobial compositions, such as but not limited to a spray bottle, a pump bottle, a nebulizer, or a dropper bottle including a medicine dropper. A kit can for example comprise a first container having a composition formulated for topical administration as a nasal spray, wherein the first container is a spray bottle, and a second container having a composition formulated for topical administration as dental drops, wherein the second container is a dropper bottle. Alternatively, the kit may comprise a container having a composition formulated for topical administration, wherein the container is a bottle capable of delivering the composition as a nasal spray and as dental drops. Any kit can further include and instructions for preparing, administering and/or delivering any of the antimicrobial compositions according to any of the methods described herein.

In another aspect, the present disclosure provides a method of treating a pharyngeal condition in a subject. The method comprises inhibiting microbial adherence to pharyngeal surface tissue in the subject by contacting the surface tissue with a composition comprising an antimicrobial effective concentration of sucralose, which can be any of the antimicrobial compositions described herein.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

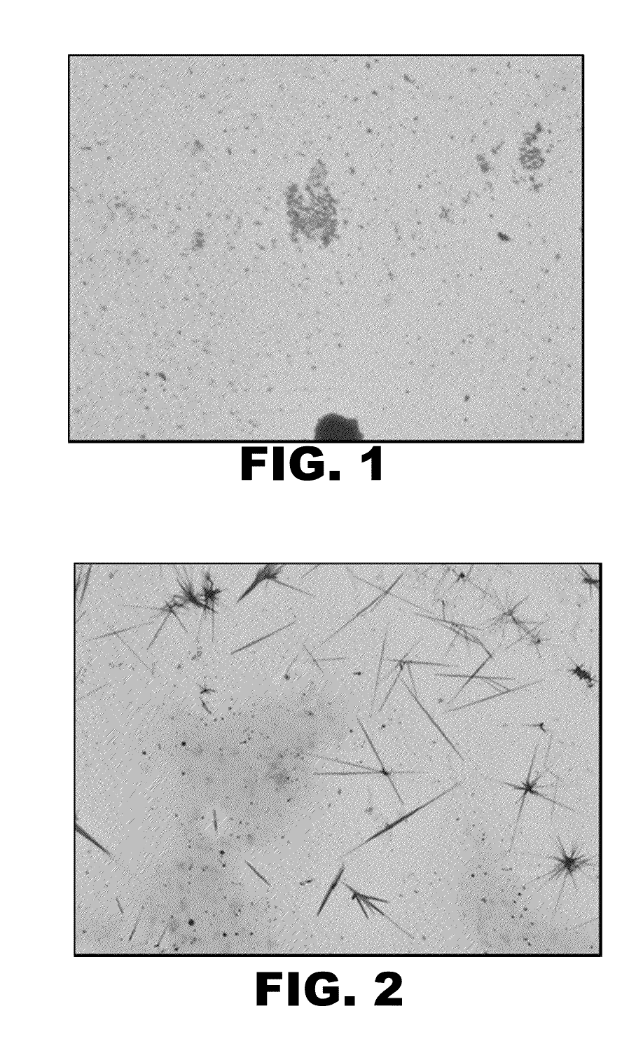

FIG. 1 is a photomicrograph (100×) of a biofilm grown on a glass substrate from a human saliva sample, following treatment with a control (sucrose) solution and before shear force application.

FIG. 2 is a photomicrograph (100×) of a biofilm grown on a glass substrate from a human saliva sample, following treatment with a test (sucralose) solution and before shear force application.

FIG. 3 is a photomicrograph (100×) of a biofilm grown on a glass substrate from a human saliva sample, following treatment with a control (sucrose) solution and after shear force application.

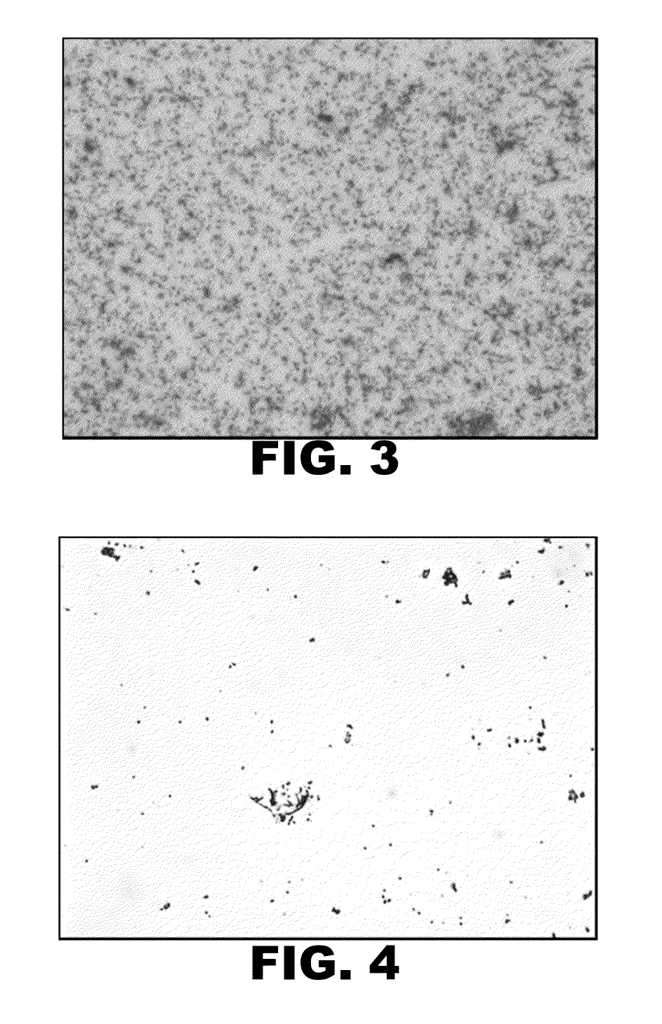

FIG. 4 is a photomicrograph (100×) of a biofilm grown on a glass substrate from a human saliva sample, following treatment with a test (sucralose) solution and after shear force application.

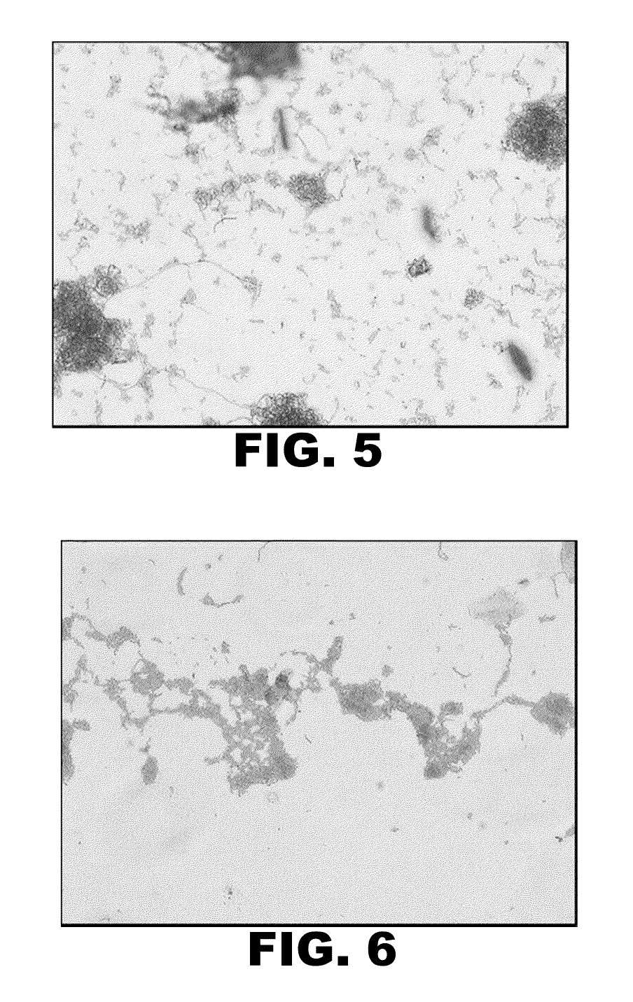

FIG. 5 is a photomicrograph (bright field, 400×) of a biofilm grown on a glass substrate from two human saliva samples, 0 minutes after mixing the samples with a test (sucralose) solution on the slide.

FIG. 6 is a photomicrograph (bright field, 400×) of a biofilm grown on a glass substrate from the two human saliva samples, 40 minutes after mixing the samples with a test (sucralose) solution on the slide.

FIG. 7 is a photomicrograph (bright field, 400×) of a biofilm grown on a glass substrate from a single human saliva sample from a volunteer with a prior history of use of a dental drop formulation of a sucralose composition, prior to in vitro exposure of the biofilm to a test (sucralose) solution.

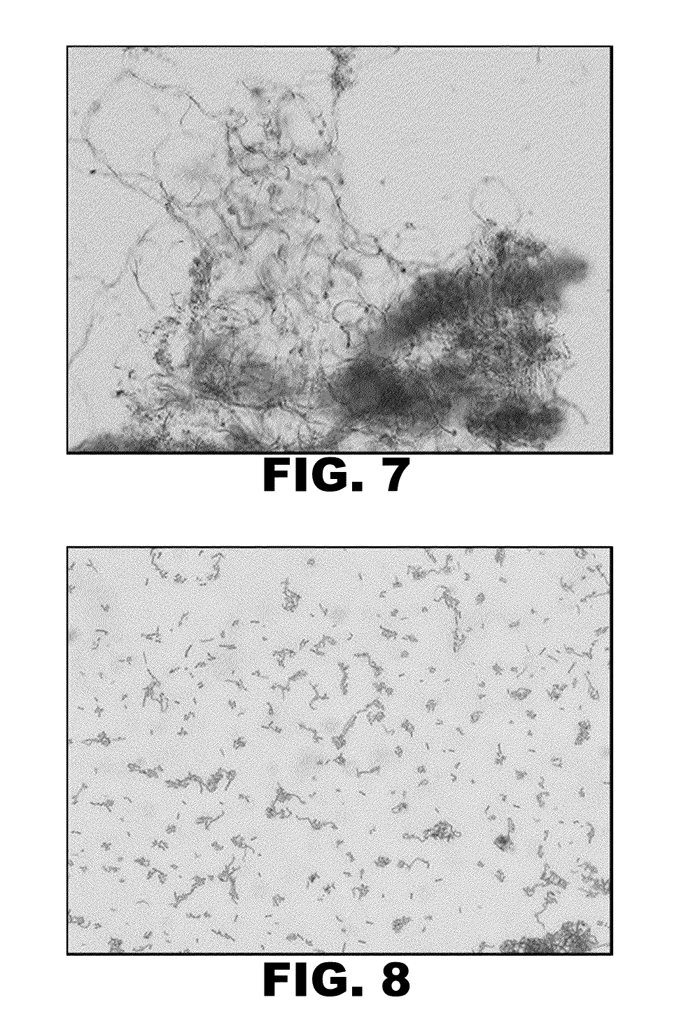

FIG. 8 is a photomicrograph (bright field, 400×) of a biofilm grown on a glass substrate from a single human saliva sample, from a volunteer with no prior history of use of a sucralose solution, prior to in vitro exposure to the test (sucralose) solution.

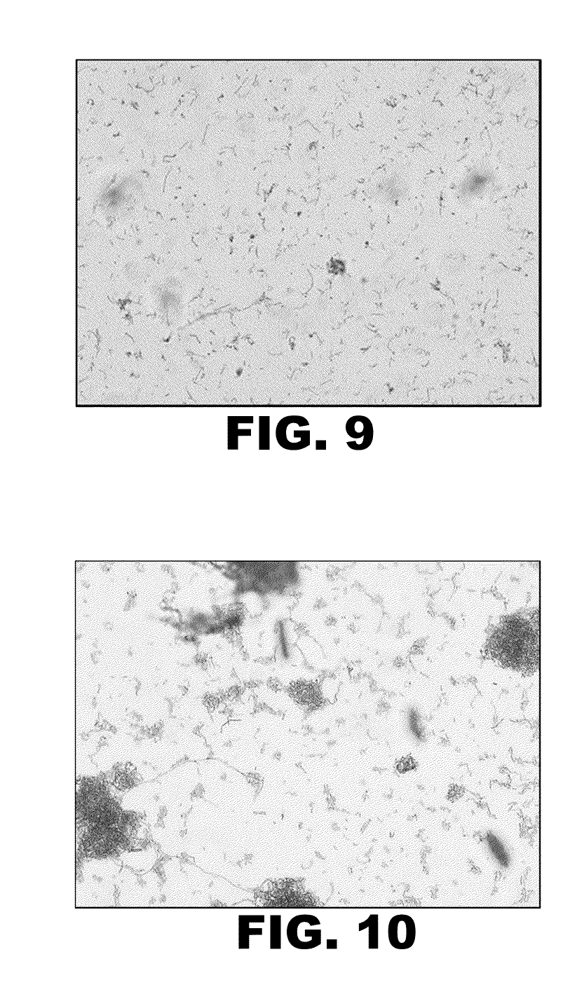

FIG. 9 is a photomicrograph (bright field, 400×) of a biofilm grown on a glass substrate from a single human saliva sample, from a volunteer with a prior history of use of a dental drop formulation of a sucralose composition, 40 minutes after in vitro exposure to the test (sucralose) solution.

FIG. 10 is a photomicrograph (bright field, 400×) of a biofilm grown on a glass substrate from a single human saliva sample from a volunteer with no prior history of use of a sucralose solution, prior to in vitro exposure to the test (sucralose) solution.

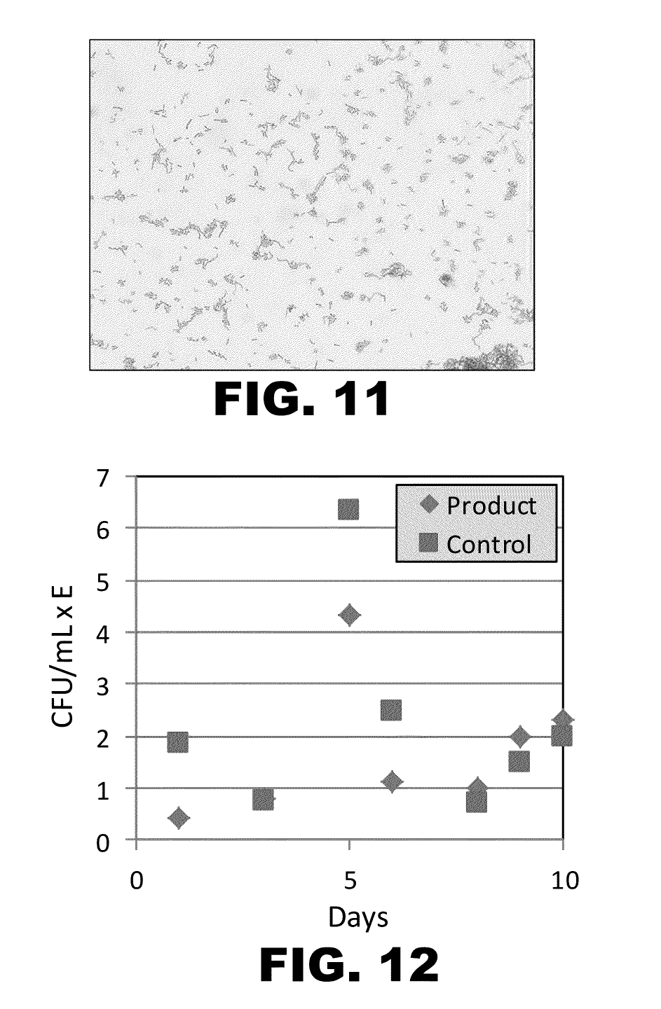

FIG. 11 is a photomicrograph (bright field, 400×) of a biofilm grown on a glass substrate from a single human saliva sample from a volunteer with a prior history of use of a dental drop formulation of a sucralose solution, prior to in vitro exposure to the test (sucralose) solution.

FIG. 12 is a culture growth curve showing the difference in culture growth (CFU/mL×E) over 10 days for a culture exposed to a test (sucralose) solution and a control culture.

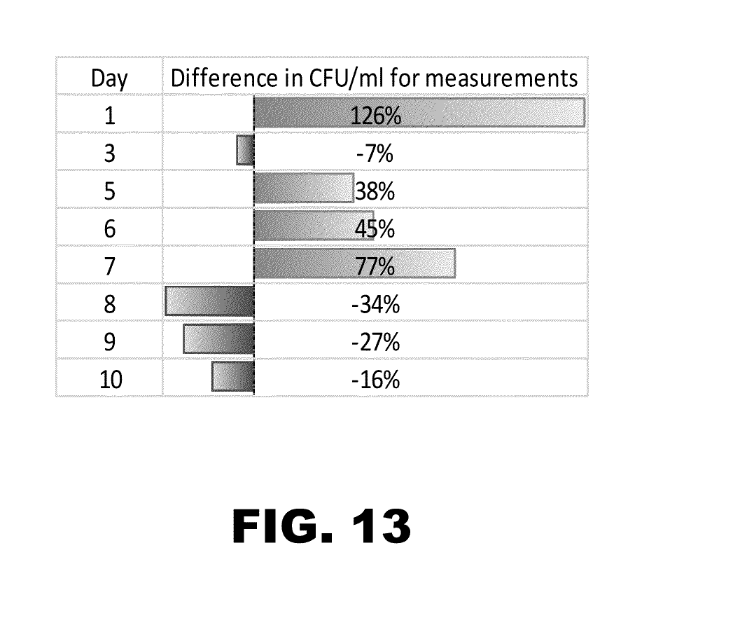

FIG. 13 is a table showing the difference in culture growth for control and product whole saliva samples.

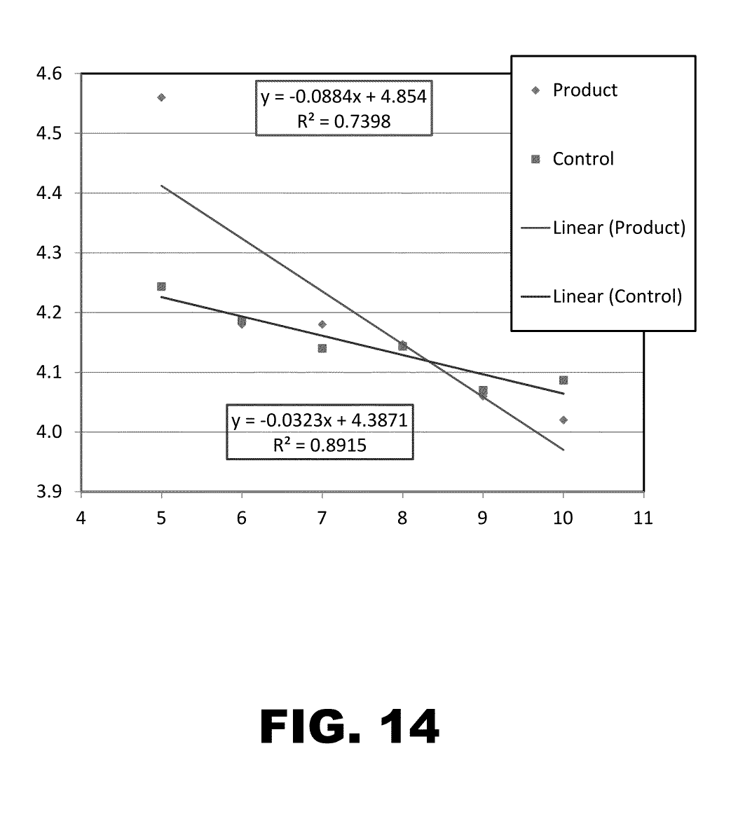

FIG. 14 is a graph of culture media growth for whole saliva samples, test and control.

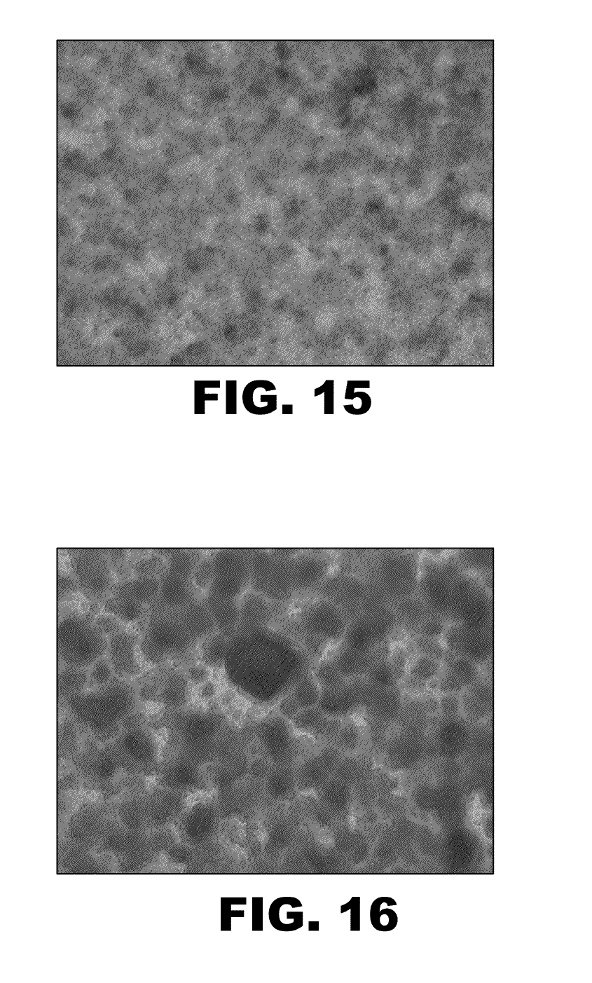

FIG. 15 is a photomicrograph (bright field, 100×), no in vivo product use, before shear force applied, of a biofilm grown on a glass substrate.

FIG. 16 is a photomicrograph (bright field, 100×), no in vivo product use, after shear force applied, of a biofilm grown on a glass substrate.

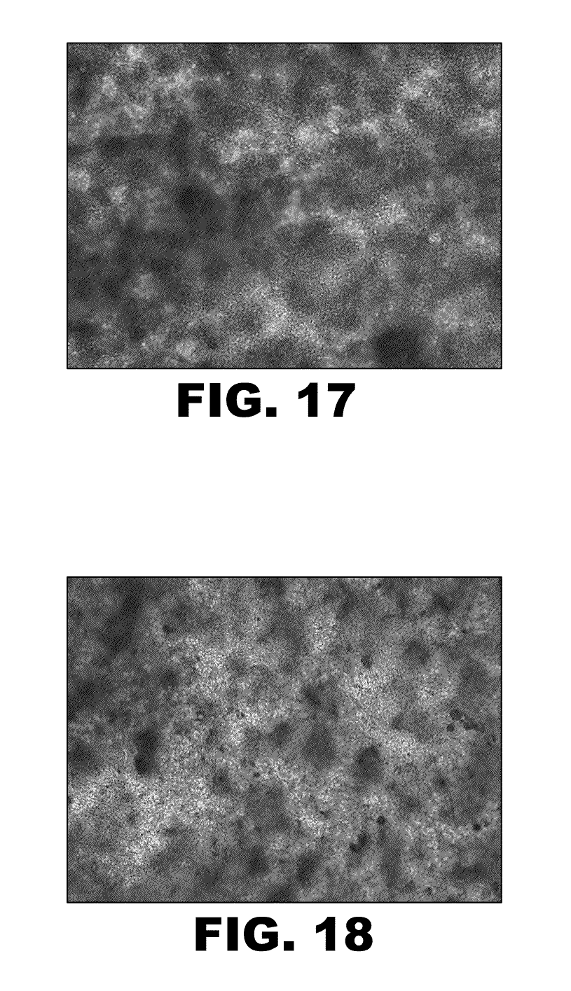

FIG. 17 is a photomicrograph (bright field, 100×), in vivo product use, before shear force applied, of a biofilm grown on a glass substrate.

FIG. 18 is a photomicrograph (bright field, 100×), in vivo product use, after shear force applied, of a biofilm grown on a glass substrate.

FIG. 19 is a photomicrograph (bright field, 400×), no in vivo product use, before shear force applied, of a biofilm grown on a glass substrate.

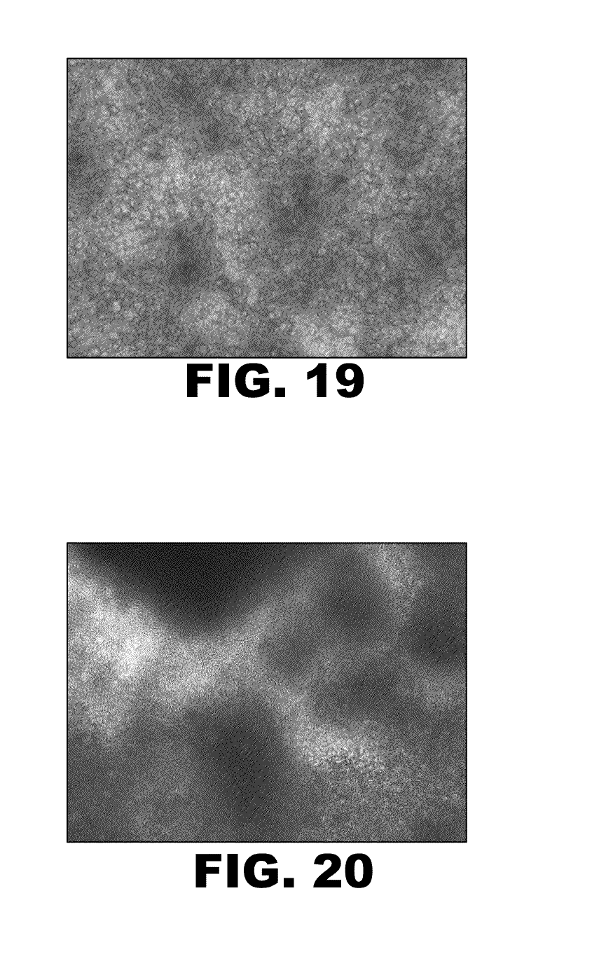

FIG. 20 is a photomicrograph (bright field, 400×), no in vivo product use, after shear force applied, of a biofilm grown on a glass substrate.

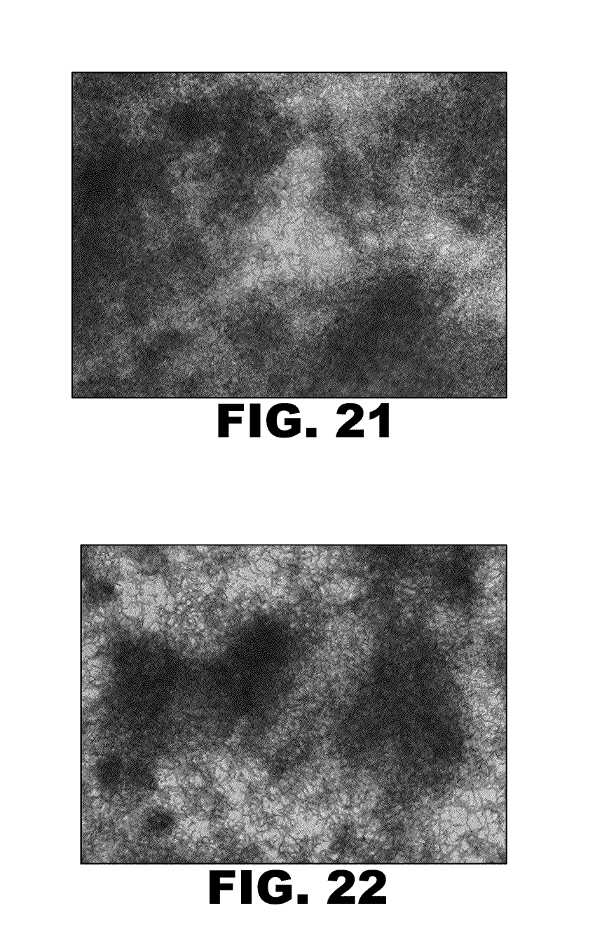

FIG. 21 is a photomicrograph (bright field, 400×), in vivo product use, before shear force applied, of a biofilm grown on a glass substrate.

FIG. 22 is a photomicrograph (bright field, 400×), in vivo product use, after shear force applied, of a biofilm grown on a glass substrate.

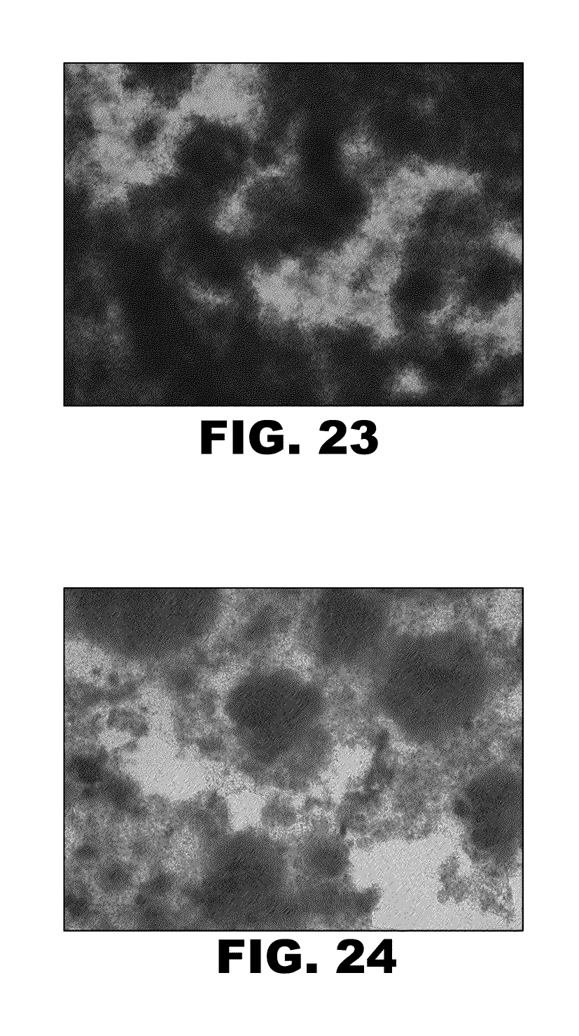

FIG. 23 is a photomicrograph (bright field, 100×), no in vivo product use, 22 hrs in vitro product exposure, before shear force applied, of a biofilm grown on a glass substrate.

FIG. 24 is a photomicrograph (bright field, 100×), no in vivo product use, 22 hrs in vitro product exposure, after shear force applied, of a biofilm grown on a glass substrate.

FIG. 25 is a photomicrograph (bright field, 100×), in vivo product use, 22 hrs in vitro product exposure, before shear force applied, of a biofilm grown on a glass substrate.

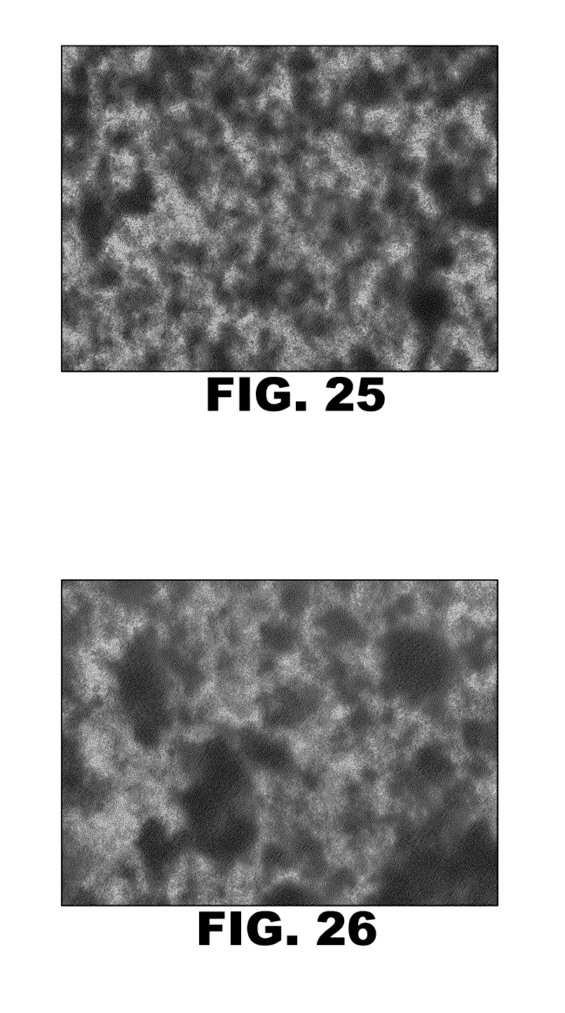

FIG. 26 is a photomicrograph (bright field, 100×), in vivo product use, 22 hrs in vitro product-exposure, after shear force applied, of a biofilm grown on a glass substrate.

FIG. 27 is a photomicrograph (bright field, 400×), no in vivo product use, 22 hrs in vitro product exposure, before shear force applied, of a biofilm grown on a glass substrate.

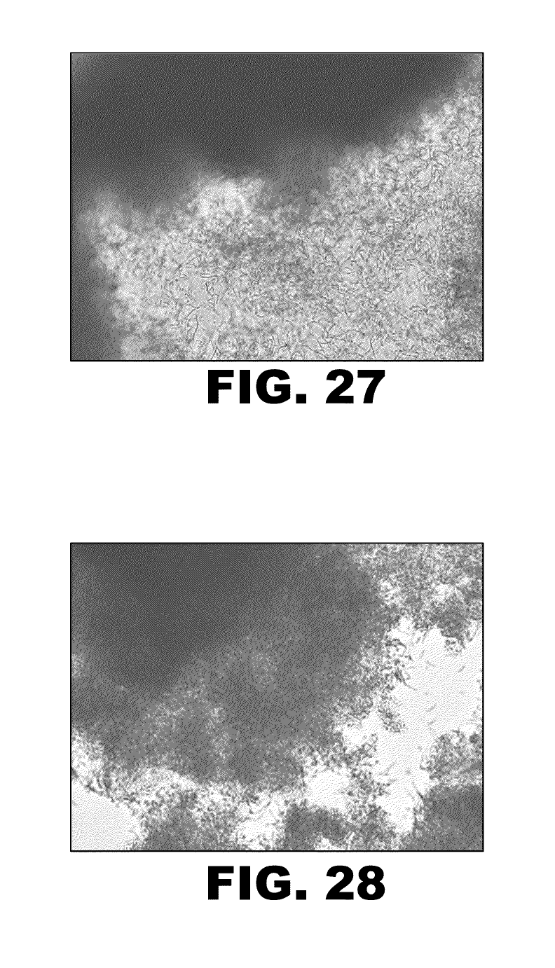

FIG. 28 is a photomicrograph (bright field, 400×), no in vivo product use, 22 hrs in vitro product exposure, after shear force applied, of a biofilm grown on a glass substrate.

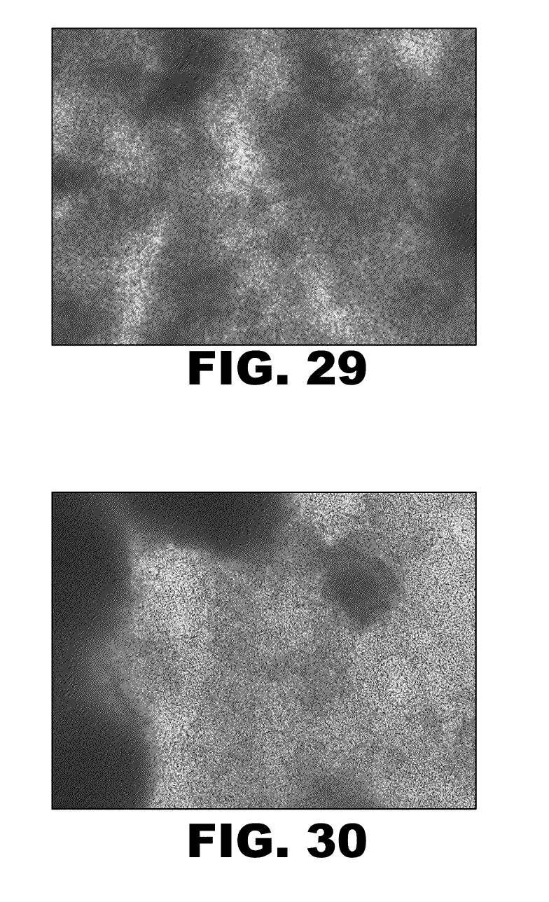

FIG. 29 is a photomicrograph (bright field, 400×), in vivo product use, 22 hrs in vitro product exposure, before shear force applied, of a biofilm grown on a glass substrate.

FIG. 30 is a photomicrograph (bright field, 400×), In vivo product use, 22 hrs in vitro product exposure, after shear force applied, of a biofilm grown on a glass substrate.

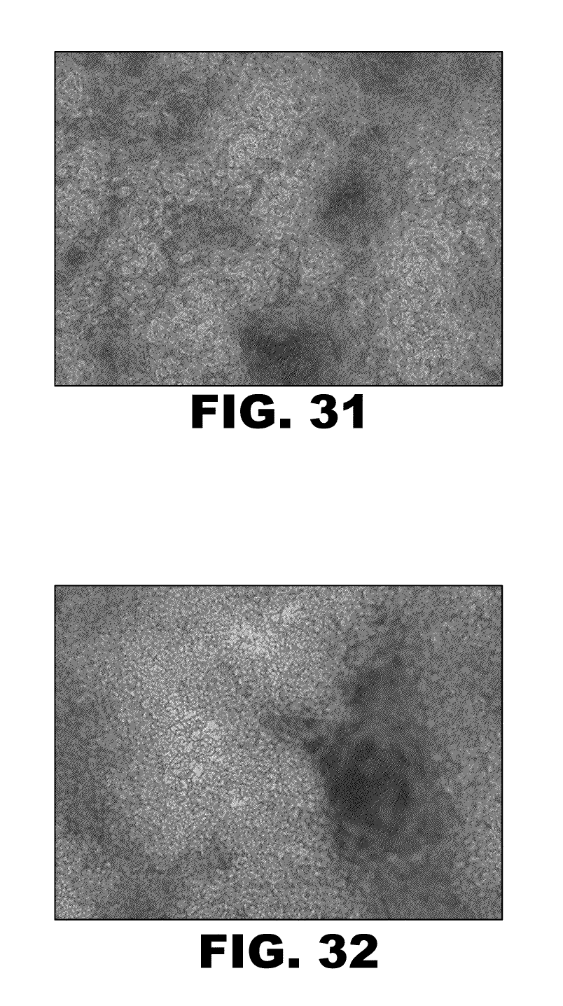

FIG. 31 is a photomicrograph (bright field, 400×), in vivo product use, 22 hrs in vitro product exposure, after shear force applied, phase contrast, 400× magnification. Biofilm peak (blue), of a biofilm grown on a glass substrate.

FIG. 32 is a photomicrograph (bright field, 400×), in vivo product use, 22 hrs in vitro product exposure, after shear force applied. Biofilm peak (blue, at right) rises above an area of biofilm thin enough to allow light through (pink), of a biofilm grown on a glass substrate.

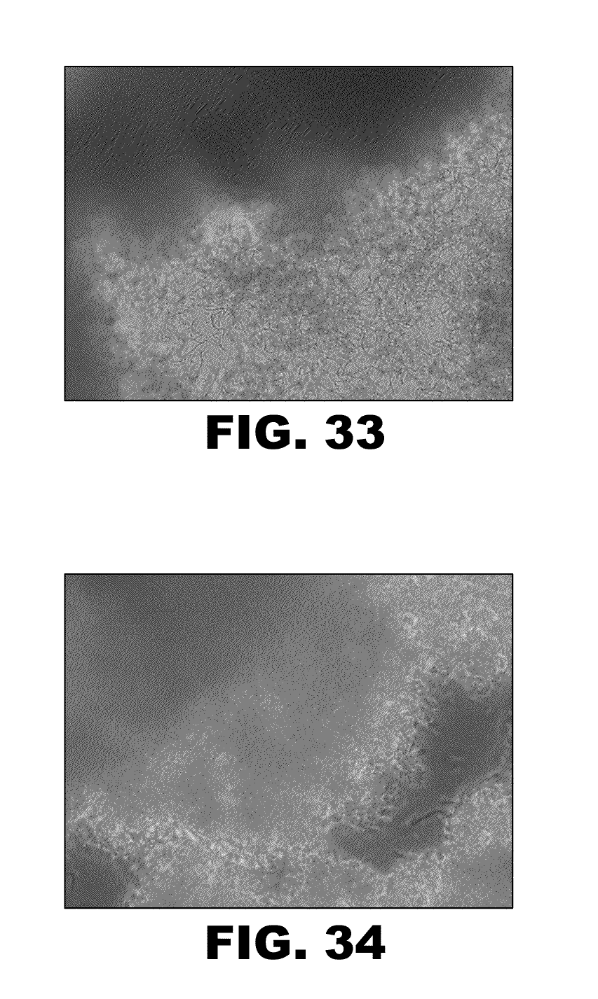

FIG. 33 is a photomicrograph (bright field, 400×), no in vivo product use, 22 hrs in vitro product exposure, before shear force applied, phase contrast, of a biofilm grown on a glass substrate.

FIG. 34 is a photomicrograph (bright field, 400×), no in vivo product use, 22 hrs in vitro product exposure, after shear force applied, phase contrast, of a biofilm grown on a glass substrate.

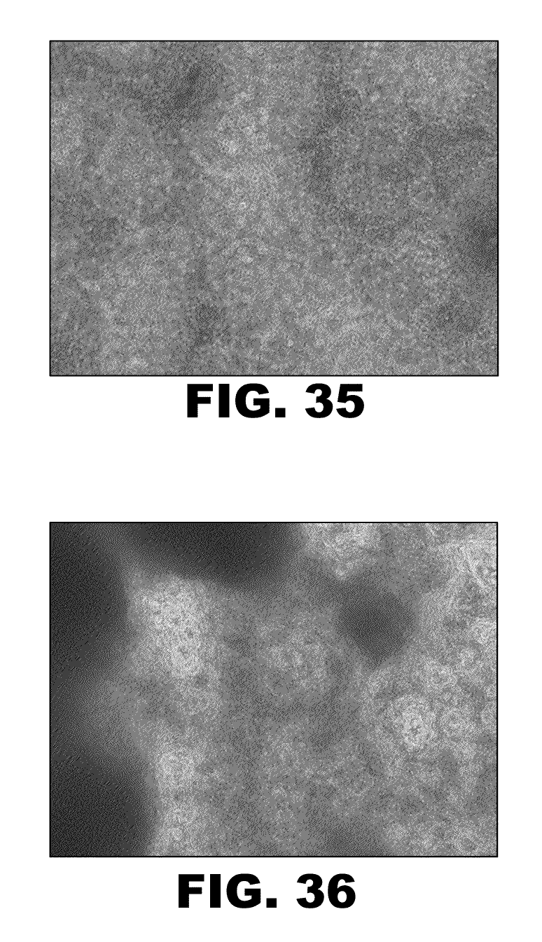

FIG. 35 is a photomicrograph (bright field, 400×), in vivo product use, 22 hrs in vitro product exposure, before shear force applied, phase contrast, of a biofilm grown on a glass substrate.

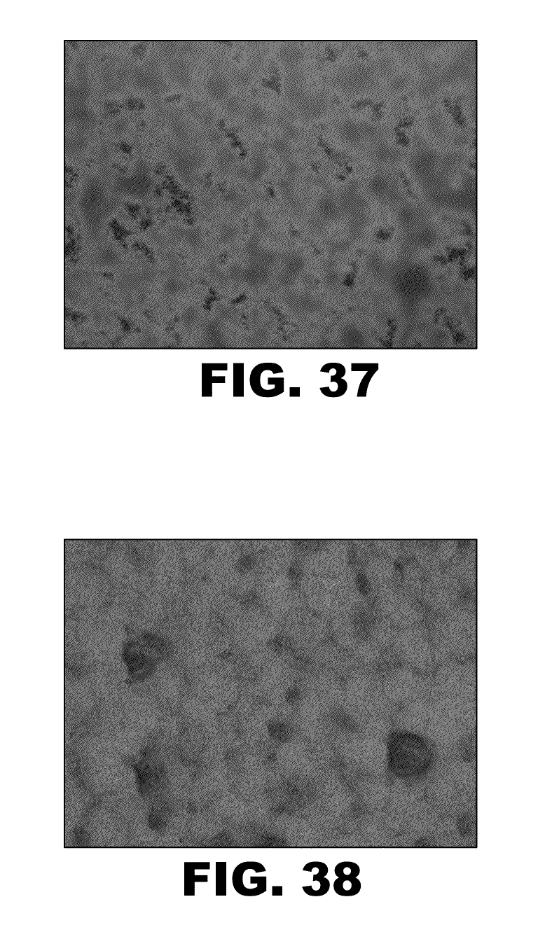

FIG. 37 is a photomicrograph (bright field, 100×), in vivo product use, 22 hrs in vitro product exposure, before shear force applied, phase contrast, of a biofilm grown on a glass substrate.

FIG. 36 is a photomicrograph (bright field, 400×), in vivo product use, 22 hrs in vitro product exposure, after shear force applied, phase contrast, of a biofilm grown on a glass substrate.

FIG. 38 is a photomicrograph (bright field, 100×), in vivo product use, 22 hrs in vitro product exposure, after shear force applied, phase contrast, of a biofilm grown on a glass substrate.

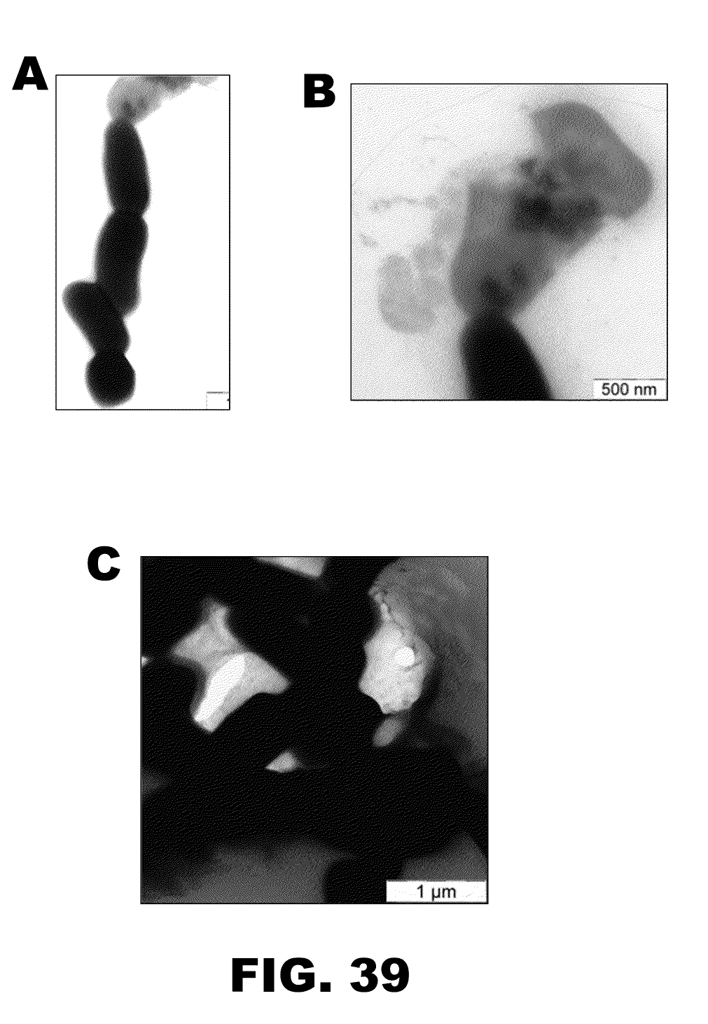

FIG. 39A-C TEM image, in vivo product exposure showing lysed cell FIGURE A and B (left, middle) and holes in biofilm matrix FIGURE C (right).

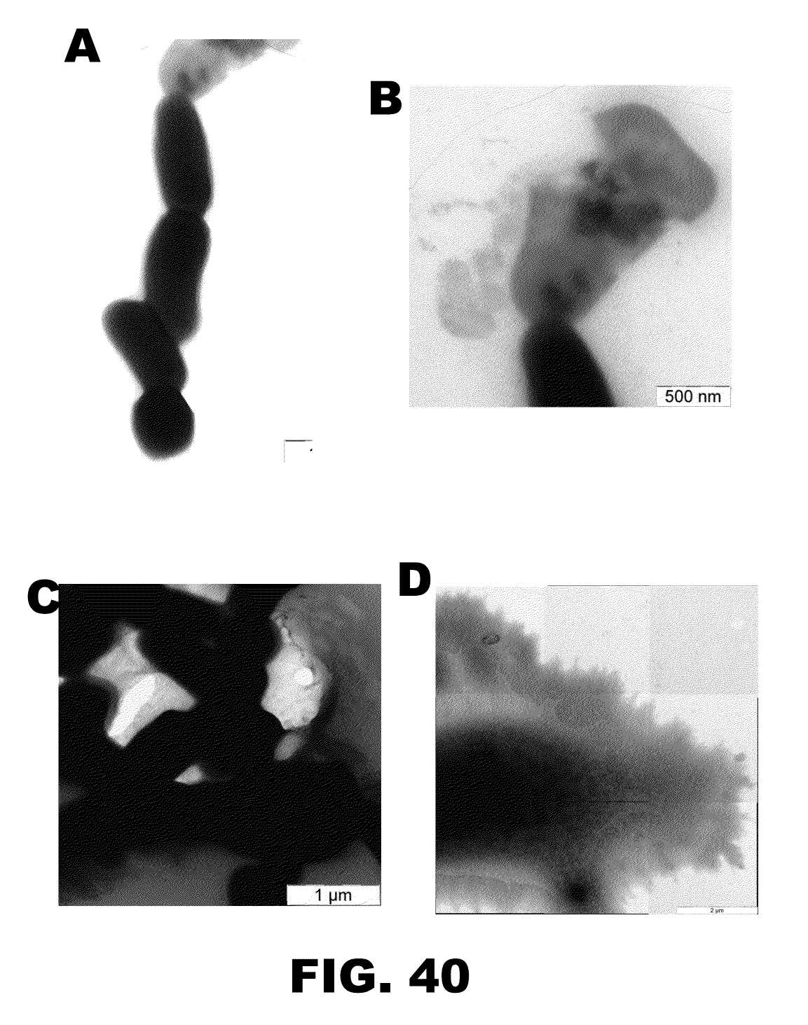

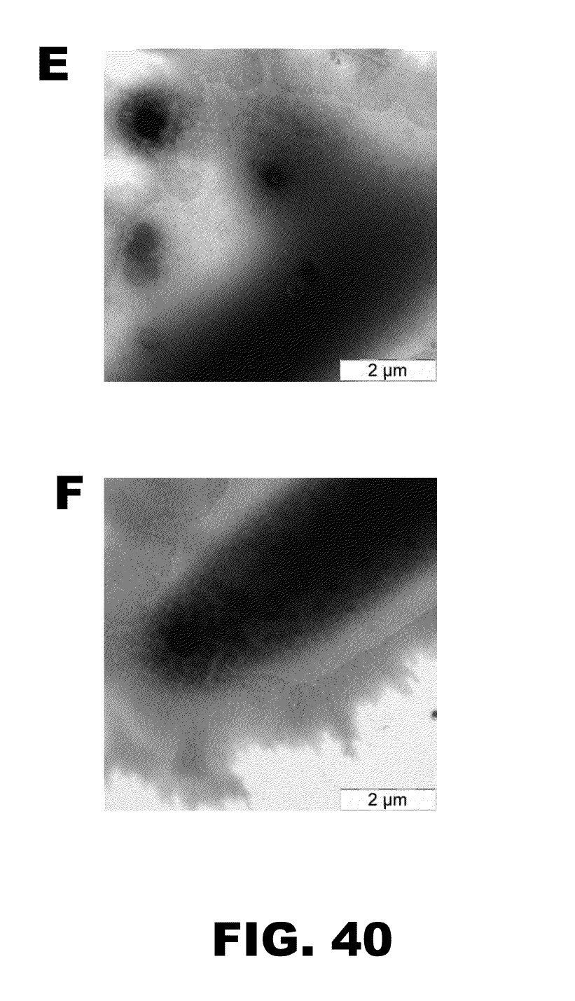

FIG. 40A-F TEM image, in vivo control (no product exposure).

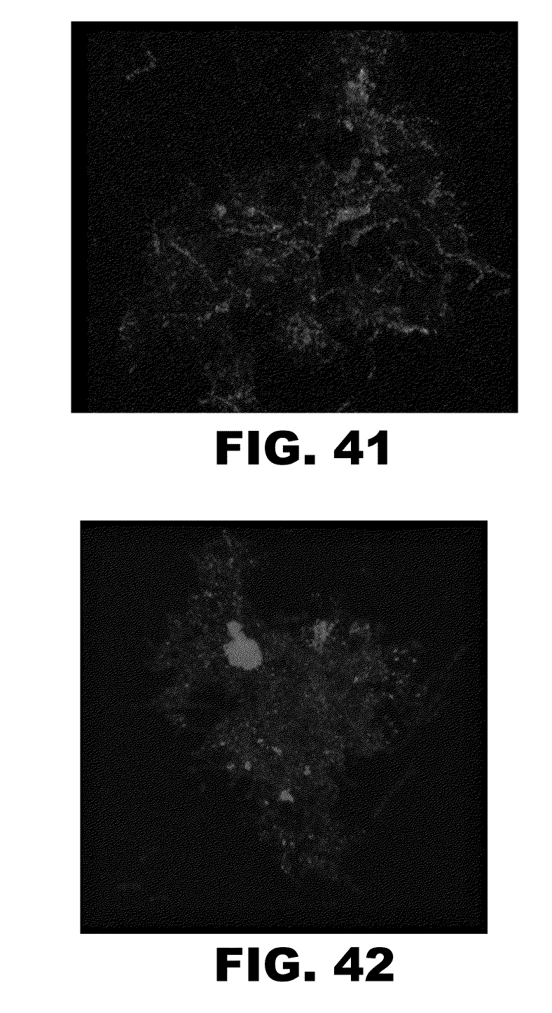

FIG. 41 is a photomicrograph of a confocal microscopy image, in vitro exposure only, no in vivo exposure (15 hours in vitro exposure time). Calcophor (red) and Syto (green) dye.

FIG. 42 is a photomicrograph of a confocal microscopy image, with no in vivo or in vitro product exposure. Calcophor (red) and Syto (green) dye.

### DETAILED DESCRIPTION OF THE INVENTION

Unless otherwise defined herein, scientific and technical terms used in connection with the present disclosure shall have the meanings that are commonly understood by those of ordinary skill in the art. For example, any nomenclatures used in connection with, and techniques of oral hygiene product manufacturing described herein are those that are well known and commonly used in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities and plural terms shall include the singular. Standard techniques are used for chemical syntheses, chemical analyses, pharmaceutical preparation, formulation, delivery, diagnosis and treatment of all subjects, human and animal.

For the recitation of numeric ranges herein, each intervening number there between with the same degree of precision is explicitly contemplated. For example, for the range 6-9, the numbers 7 and 8 are expressly contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “antimicrobial” as used herein encompasses both microbiocidal activity wherein microbes are killed and/or the number of viable microbes reduced, and microbiostatic activity, wherein the proliferation or growth of microbes is inhibited, reduced or eliminated.

### A. Compositions

It was surprisingly discovered that compositions comprising certain concentrations of sucralose, in contrast to other sugar substitutes such as xylitol, have an antimicrobial effect on tissue, when present in a sucrose-free in vivo environment for sustained periods of time. Preferably, the compositions are present for at least about 6-8 or more hours per day, and consistently over a period of at least about 1-2 weeks, and preferably longer, at least about 1 month, 2 months or 3 or more months. The compositions and methods described herein may be used to treat a variety of microbial conditions related to colonization of bodily tissue by bacteria. Among such conditions that can benefit from the compositions and methods described herein are pharyngeal conditions, but also ophthalmological and skin conditions.

While not wishing to be bound by theory, it is believed that antimicrobial concentrations of sucralose inhibit microbial adherence to tissue, such as pharyngeal surface tissue, by creating a pharyngeal environment starved of sucrose, thereby inhibiting binding and import of sucrose into bacteria, and inhibiting formation of extra-cellular polysaccharides by bacterial glucosyltransferase, which uses sucrose as a substrate for synthesis of the adhesive extra-cellular polysaccharides used by bacteria for adhesion to surface tissue. Inhibiting microbial adherence to pharyngeal surface tissue inhibits an essential first step in establishing a pathogenic relationship between commensal pharyngeal microorganisms and the host. Alternatively, antimicrobial concentrations of sucralose may disrupt bacterial fimbriae chemosensation, or disrupt the bacterial capsule or of the extracellular polysaccharide matrix of pathogenic oral bacteria. Changes in biofilm with continuous product use suggest that the present disclosure compositions permeate biofilm volume, weaken its structure, and reduce its adhesive properties. These changes include removal of biofilm with reduced shear force, absence of pellicle from enamel surfaces, and reduction of plaque and tarter at the gum line. Overall, it has been surprisingly found that the compositions described herein, when used as described herein, demonstrate antimicrobial function highly useful in treating and controlling a variety of pharyngeal conditions arising from bacterial colonization.

As such, the present disclosure provides compositions comprising antimicrobial concentrations of sucralose and methods of using such compositions for treating pharyngeal conditions. Importantly, because antimicrobial concentrations of sucralose are intended for application in a pharyngeal environment starved of sucrose, compositions of the present disclosure should be substantially free of sucrose, and preferably free of sucrose. Bacteria do not distinguish between sucralose and sucrose. If both compounds (sucralose and sucrose) are present and sucrose dominates, then the bacteria are diminished, but not eliminated. In an exemplary method, the sucralose is administered during the quiescent period during sleep when little or no sugar is available for consumption, to achieve maximum exposure of bacteria to sucralose. Advantageously, using compositions comprising antimicrobial concentrations of sucralose for pharyngeal health eliminates and then prevents further adhesion of bacteria to pharyngeal tissue, thereby reducing the population of potentially pathogenic bacteria in the pharynx by 95% or more preventing colonization, preventing inflammation of tissue, and treating pharyngeal conditions without resorting to the use of antibiotics which can lead to antimicrobial resistance, digestive tract pain, allergic reaction, discomfort and diarrhea. Compositions comprising sucralose and methods of using such compositions are described below.

The present disclosure encompasses a composition comprising an antimicrobial effective concentration of a chlorinated sucrose derivative (chlorosucrose). A preferred chlorosucrose is sucralose. Sucralose is a chlorosucrose sweetener having the structure of Formula (I).

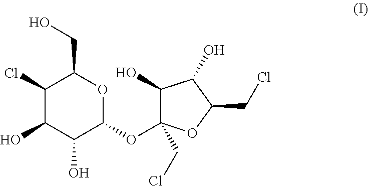 [SMILES: OC[C@H]1O[C@H](O[C@]2(CCl)O[C@H](CCl)[C@@H](O)[C@@H]2O)[C@H](O)[C@@H](O)[C@H]1Cl]

Compositions of the disclosure comprise an antimicrobial effective concentration of sucralose. An antimicrobial effective concentration of sucralose comprises at least about 1% w/w sucralose or more. For instance, compositions of the disclosure may comprise about 1, 5, 10, 15, 20, or about 25% sucralose or more. Preferably, a composition of the disclosure comprises about 1 to about 25% sucralose. More preferably, a composition of the disclosure comprises about 1 to about 5% sucralose. Even more preferably, a composition of the disclosure comprises about 1, 2, 3, 4, or about 5% sucralose. While a composition as described herein can contain more than about 25% w/w sucralose, without wishing to be bound by theory, it is believed that a composition that contains amounts of sucralose over about 25% w/w can contribute to disruption of gut bacteria, which has been linked to gastrointestinal diseases such as irritable bowel disease and lupus. As such, it is preferred that the compositions described herein contain no more than about 25% w/w sucralose. Amounts of sucralose refer to pure sucralose, which is commercially available in liquid or solid (powdered) form, for example from Tate and Lyle (www.tateandlyle.com).

Compositions may be formulated to be compatible with their intended route of administration. Examples of routes of administration include parenteral, e.g., intravenous, intradermal, subcutaneous, oral (e.g., inhalation), transdermal, topical, transmucosal, and rectal administration. Formulation of compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Preferably, compositions of the disclosure are formulated for topical administration to the oral cavity, i.e., to any pharyngeal surface tissue. Such compositions can comprise for example an antimicrobial effective amount of sucralose and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with sucralose, use thereof in the compositions is contemplated. Supplementary active compounds may also be incorporated into the compositions.

Generally, compositions of the disclosure are formulated for topical administration on surface tissue in the pharynx. The pharynx makes up the part of the throat situated immediately posterior to the nasal cavity, posterior to the mouth and superior to the esophagus and larynx. The human pharynx is conventionally divided into three sections: the nasopharynx, which extends from the base of the skull to the upper surface of the soft palate and includes the space between the internal nares and the soft palate and lies above the oral cavity, and the pharyngeal tonsils; the oropharynx, which extends from the uvula into the mouth, and includes the base of the tongue, the tonsil, and the inferior surface of the soft palate; and the laryngopharynx, which is the part of the throat that connects to the esophagus. Preferably, compositions of the disclosure are formulated for topical administration on surface tissue in the oropharynx and the nasopharynx.

A composition of the present disclosure formulated for topical administration on pharyngeal surfaces is a composition which, during the normal course of usage, is not intentionally swallowed but is rather retained on pharyngeal surfaces for a time sufficient to contact substantially all of the pharyngeal surfaces. Examples of compositions that may be formulated for topical administration on pharyngeal surfaces may include, but are not limited to: toothpaste, tooth gels, dentifrice, edible film, mouthwash or mouth rinses, topical oral gels, foam, varnish, denture cleanser, an oral spray, drops, a mist, dental floss, confectionery including chewing gum and lozenge, and the like. Preferably, compositions are formulated as known in the art for topical administration on pharyngeal surfaces as a mouth wash or mouth rinse, a topical oral gel, a lozenge, nasal drops, dental drops, a mist, a nasal spray, or a mouth spray. More preferred are compositions formulated for topical administration on pharyngeal surfaces as nasal drops, dental drops, a mist, a nasal spray, a mouth wash or rinse, or a mouth spray. For such preferred methods of administration, compositions of the disclosure may be filled in containers for the storage and administration of drops, sprays, or mist, e.g. metered-dose spray devices, devices for sprays, nebulizers, squeeze bottles, or bottles for drops.

Such preferred formulations for topical administration are normally prepared as buffered saline aqueous solutions. As such, in addition to sucralose, compositions of the present disclosure may comprise a tonicity-adjusting agent such as sodium chloride. Compositions may comprise sodium chloride in an amount sufficient to cause the final composition to have an osmolality acceptable for the intended pharyngeal tissue. A nasally and orally acceptable osmolality is preferably 240-350 mOsm/kg. Most preferably, the amount of sodium chloride in compositions of the present disclosure is an amount sufficient to cause the compositions to have an osmolality of 260-330 mOsm/kg. As such, compositions may comprise 0.3-0.9% sodium chloride. Preferably, compositions may comprise 0.35-0.55% sodium chloride, and in a most preferred embodiment, the compositions may comprise 0.35-0.45% sodium chloride.

Compositions of the present disclosure may also comprise a pharmaceutically acceptable pH-adjusting agent. Such pH-adjusting agents are known and include, but are not limited to, acetic acid, lactic acid, sodium lactate, potassium lactate, calcium lactate, citric acid, tartaric acid, sodium phosphates, sodium biphosphates, potassium phosphates, calcium phosphates, magnesium phosphates, adipic acid, succinic acid, sodium fumarate, potassium fumarate, calcium fumarate, hydrogen carbonate (bicarbonate), and potassium carbonate. Generally, compositions of the present disclosure comprise an amount of pH-adjusting agent sufficient to obtain a neutral composition pH. Preferably, compositions are buffered using bicarbonate at a concentration of about 0.05 to about 0.5%, preferably at a concentration of about 0.1% to about 0.2%.

Compositions of the present disclosure may also comprise one or more sugar alcohols such as, in non-limiting example, xylitol. Because xylitol is believed to kill bacteria by a different mechanism than sucralose (ingestion versus preventing adhesion or other mechanism), xylitol and sucralose can be combined in a composition to provide dual modes of anti-bacterial function. Other sugar alcohols suitable for combining with sucralose in a composition of the present disclosure include other non-toxic sugar alcohols, such as sorbitol, erythritol, malitol and the like.

Compositions may be sterile and may comprise amounts of a preservative and/or a chelating agent considered in the art to be effective to avoid microbial contamination. Additionally, compositions may be prepared as sterile compositions and packaged in containers to avoid microbial contamination and may comprise a preservative and/or a chelating agent such that the compositions pass United States Pharmacopeia/National Formulary criteria for antimicrobial effectiveness, and more preferably the Pharm. Eur. 5th Edition criteria for antimicrobial preservation (Pharm. Eur. B preservative effectiveness standard). The term “effective amount” as used herein is used to describe an amount of a compound, component, or composition sufficient to significantly induce a positive benefit, but low enough to avoid serious side effects, i.e., to provide a reasonable benefit to risk ratio, within the scope of sound medical judgment. Non-limiting examples of suitable chelating agents include ethylenediaminetetraacetic acid (EDTA) and the like. The amount of EDTA in compositions of the present disclosure is preferably 0.005-0.015%, and more preferably 0.01%. Non-limiting examples of preservative ingredients that may be used in a composition of the present disclosure include p-hydroxybenzoic acid ester, benzalkonium chloride, benzododecinium bromide, phenyl-carbinol, sodium sorbate, potassium sorbate, calcium sorbate, benzoic acid, sodium benzoate, potassium benzoate, calcium benzoate, propyl paraben, methyl paraben, dimethyl dicarbonate, sodium propionate, calcium propionate, potassium propionate, and calcium disodium ethylenediaminetetraacetate. A most preferred preservative is benzalkonium chloride. Another most preferred preservative is phenyl-carbinol.

Compositions may optionally further comprise safe and effective amounts of desensitizing agents, flavorings, colorings, fluoride sources, emulsifiers and other additives such as remineralizing agents. Emulsifiers may include, without limitation, lecithin, sodium citrates, sodium phosphates, potassium phosphates, calcium phosphates, polyethylene, and polysorbate 80.

Non-limiting examples of fluoride sources are sodium fluoride and stannous fluoride. Application of fluoride ions to dental enamel serves to protect teeth against decay. A wide variety of fluoride ion-yielding materials can be employed as sources of soluble fluoride in the present compositions. Examples of suitable fluoride ion-yielding materials are found in U.S. Pat. No. 3,535,421, Oct. 20, 1970, issued to Briner et al., and U.S. Pat. No. 3,678,154, Jul. 18, 1972, issued to Widder et al. Preferred fluoride ion sources for use herein include sodium fluoride, potassium fluoride, stannous fluoride, ammonium fluoride and mixtures thereof. Sodium fluoride is an exemplary fluoride ion source.

A remineralizing agent can be included in any composition. Non-limiting examples of a remineralizing agent are hydroxylapatite (also called “hydroxyapatite, “HA”) and calcium phosphate. For example, hydroxylapatite may be included as an additive for remineralizing tooth enamel. HA generates mineral ions needed for remineralization. A composition as described herein and which includes a remineralizing agent such as HA accelerates the process of remineralization by reducing lactic acid and raising salivary pH. Generally, use of relatively expensive HA in oral hygiene compositions or dentifrices is not considered cost effective, because the amount of HA needed is fairly substantial in view of the quantity that is rinsed out of the mouth in typical use. In contrast, the present compositions are retained within the oral cavity and as such, a lower amount of HA can be effective.

Examples of flavoring agents that may be contained in the composition, include, without limitation, wintergreen oil, oregano oil, bay leaf oil, peppermint oil, anethole, spearmint oil, clove oil, sage oil, sassafras oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, methyl salicylate, vanillin, eugenol, furaneol, linalool, menthol, thymol, cinnamaldehyde, citral, methyl butanoate, pentylbutanoate, pentylpentanoate, tea tree oil, pineapplemint oil, and eucalyptus oil.

Non-limiting examples of coloring agents are dyes such as FD & C blue No. 1, D & C yellow No. 10 and D & C yellow No. 3, titanium dioxide, tartrazine, chlorophyll, caramel, carotene, annatto extracts, lycopene, lutein, saffron, anthocyanins, calcium carbonate, tannic acid, erythrosine, amaranth, carmines, curcumin and riboflavin.

Examples of desensitizing agents that may be contained in the composition include, without limitation, esium nitrate, cesium citrate, stannous fluoride, potassium oxalate, strontium chloride, potassium nitrate, natural herbs such as gall nut, Asarum, Cubebin, Galanga, scutellaria, Liangmianzhen, and Baizhi. Analgesics, including low levels of non-steroidal anti-inflammatory agents, such as ketorolac, flurbinprofen, ibuprofen, naproxen, indomethacin, aspirin, ketoprofen, piroxicam and meclofenamic acid, may also be used as desensitising agents.

Compositions may optionally further comprise additional antimicrobial ingredients, and/or additional anti-calculus agents. Non-limiting antimicrobial ingredients may include: bioflavonoids, which include, without limitation, polyphenols such as gallic acid and catechin and their derivatives, theogallin, gallocatechin, epigallocatechin, epicatechin or epigallocatechin gallate; essential oils, which include, without limitation, terpene hydrocarbons, such as alpha-pinene, beta-pinene, p-cymene, limonene, aromadendrene, 1,8-cineole, terpinolene, alpha-terpineol, alpha-terpinene, gamma-terpinene, terpinen-4-ol, alloocimene, delta-3-carene, dertol, dertosol or dipentene; oxygenated terpenes, which include, without limitation, terpinen-5-ol; Lavandula officinalis; Citrus limon; Commiphora pyrrha; Pogostemon patchouli; Mentha piperita; Rosemarinus officinalis; Eucalyptus globules or Mentha arvensis; quaternary ammonium compounds, which include, without limitation, cetylpyridium chloride; bis-phenols, which include, without limitation, triclosan; and bigualides, which include, without limitation, chlorhexidine. The following essential oils are also known to have anti-microbial activity and are therefore optionally used in compositions of the present disclosure. These oils include thymol, geraniol, carvacrol, hinokitiol, eucalyptol, catechol (particularly 4-allyl catechol) and mixtures thereof.

Non-limiting examples of additional anti-calculus agents include urea, calcium glycerophosphate, sodium trimetaphosphate, polyacrylates and other polycarboxylates such as those disclosed in U.S. Pat. No. 3,429,963 issued to Shedlovsky on Feb. 25, 1969; U.S. Pat. No. 4,304,766 issued to Chang on Dec. 8, 1981; and U.S. Pat. No. 4,661,341 issued to Benedict and Sunberg on Apr. 28, 1987; polyepoxysuccinates such as those disclosed in U.S. Pat. No. 4,846,650 issued to Bendict, Bush and Sunberg on Jul. 11, 1989; ethylenediaminetetraacetic acid as disclosed in British Patent No 490,384 dated Feb. 15, 1937; nitrilotriacetic acid and related compounds as disclosed in U.S. Pat. No. 3,678,154 issued to Widder and Briner on Jul. 18, 1972; polyphosphonates as disclosed in U.S. Pat. No. 3,737,533 issued to Francis on Jun. 5, 1973; U.S. Pat. No. 3,988,443 issued to Ploger, Schmidt-Dunker and Gloxhuber on Oct. 26, 1976; and U.S. Pat. No. 4,877,603 issued to Degenhardt and Kozikowski on Oct. 31, 1989.

Further optional components for use in the compositions include antioxidants, vitamins, whitening agents such as carbamide peroxide, and mixtures thereof. Pharmaceutically active agents may be selected from analgesics, anti-allergy agents, anti-arrhythmia agents, antibiotics, anti-caries, anti-coagulants, antidepressants, anti-diarrheal agents, anti-emetics, anti-epileptics, anti-fungals, antihistamines, anti-inflammatory agents, anti-lipidemics, antiparasitics, anti-plaque agents, anti-platelet aggregation agents, anti-pruritics, anti-pyretics, anti-stress agents, anti-tartar agents, antitumor agents, antitussives, anti-ulcer agents, anxiolytics, breath fresheners, dentifrices, hormones, muscle relaxants, sedatives, tranquilizers, and vasodilators. The active agent may comprise a palatant, nutritional supplement or pharmaceutically active agent, or any combination thereof, which is water soluble.

Preferred compositions may comprise an antimicrobial effective concentration of sucralose, wherein the antimicrobial effective concentration of sucralose is about 1% to about 25% w/w, about 0.35-0.45% sodium chloride, about 0.1% to about 0.2% hydrogen carbonate, and a preservative effective amount of benzalkonium chloride. More preferred compositions may comprise an antimicrobial effective concentration of sucralose, wherein the antimicrobial effective concentration of sucralose is about 1% to about 5% w/w, and the composition further contains 0.35-0.45% sodium chloride, about 0.1% to about 0.2% hydrogen carbonate, and a preservative effective amount of benzalkonium chloride.

### B. Methods

In another aspect, the present disclosure provides methods of treating a microbial condition, such as but not limited to a pharyngeal condition in a subject. The method comprises administering to the subject a composition comprising an antimicrobial effective concentration of sucralose, and/or delivering such a composition to a tissue surface of the subject, for example to a pharyngeal surface of the subject. A composition comprising an antimicrobial effective concentration of sucralose may be as described in Section I. Preferably, the composition contains substantially no sucrose, or no sucrose, and the composition is administered regularly, for example daily, over a period of at least about one week.

In non-limiting example, a pharyngeal condition which is an oral condition, such as but not limited to gingivitis, periodontal disease, dental caries, or halitosis can be treated as follows: the composition is administered to the oral cavity. Administration is at least once daily. Preferably, following administration the oral cavity is maintained sucrose-free for a post-administration period of at least about 5-8 hours, although longer periods of about 8-10 hours or even longer can improve the antimicrobial effect. It is believed that post-administration periods routinely lasting less than 5 hours are likely to produce some useful antimicrobial effect, but less pronounced than that achieved by maintaining a longer post-administration period of at least about 5 hours or longer.

To achieve an oral environment substantially free or free of sucrose for example, the subject may simply use routine oral hygiene methods such as tooth-brushing and/or mouth-washing to remove substantially all, or all sucrose remaining in the oral cavity, and then self-administer the composition to the oral cavity. In non-limiting example, a few drops of the composition can be applied to the tongue during and/or immediately following teeth brushing.

Alternatively, the composition can be sprayed, wiped, brushed, dropped onto or otherwise applied to a surface of the oral cavity using any other method which achieves such oral delivery. For example, by wetting the toothbrush with drops of any of the antimicrobial compositions, instead of wetting with water, the brush can be sued to deliver the composition to any surfaces in the mouth that the toothbrush reaches. Brushing with drops also exposes more gum tissue to the composition than other methods of delivery, and brushing has the further advantage of removing bacteria adhering to gum tissue.

Following administration, to avoid the introduction of sucrose into the oral environment during the post-administration period, the subject can simply avoid eating or drinking the entire period. This can be conveniently accomplished for example by administering the composition immediately before a daily sleep period of at least about 5 hours, or any daily period during which the subject routinely does not eat or drink. It should be appreciated that that some foods and drink are sucrose-free and thus may still be ingested during the post-administration period while still maintaining the oral environment at least substantially sucrose-free. Administration and preferably adherence to the post-administration period can be followed consistently over a duration of at least about 1-2 weeks, and preferably longer, at least 3 weeks, at least about 1 month, at least about 2 months or at least about 3 or more months.

The term “subject,” as used herein, refers to any animal having pharyngeal surface or equivalent that is prone to deleterious bacterial colonization. The subject may be an embryo, a juvenile, or an adult. Suitable animals include vertebrates such as mammals, birds, and reptiles. Examples of suitable mammals include, without limit, rodents, companion animals, livestock, and primates. Non-limiting examples of rodents include mice, rats, hamsters, gerbils, and guinea pigs. Suitable companion animals include, but are not limited to, cats, dogs, rabbits, hedgehogs, and ferrets. Non-limiting examples of livestock include horses, goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. An exemplary subject is a human.

Pharyngeal surface tissue is colonized by a complex multi-species microbial community that plays an important role in maintenance of health and development of pharyngeal conditions. The pharyngeal microbial community comprises microorganisms that, under certain conditions, can switch to opportunistic pathogens, initiating disease and damaging the host.

Adherence of pharyngeal microorganisms to pharyngeal surface tissue is an essential first step in establishing a pathogenic relationship between the commensal microorganisms and the host. For instance, commensal oropharyngeal mutans streptococci such as Streptococcus sobrinus and Streptococcus mutans, and Lactobacilli are naturally present in the human oral microbiota but can adhere to oropharyngeal surfaces and switch to a pathogenic relationship with the host, enabling other bacteria to colonize oropharyngeal surface tissue and form dental plaque, leading to oropharyngeal conditions such as gingivitis, periodontal disease, dental caries, and halitosis. Non-limiting examples of other bacteria that may colonize oropharyngeal surfaces as a result of adherence of commensal oropharyngeal mutans streptococci and Lactobacilli include Porphyromonas gingivalis, Treponema denticola, Nocardia spp., Fusobacterium nucleaturn, Prevotella intermedia, Actinobacillus actinomycetemcomitans, and Tannerella forsythia (also referred to as Bacteroides forsythus and Tannerella forsythensis).

Similarly, pneumococcal bacteria (Streptococcus pneumonia) and Staphylococcus epidermidis reside asymptomatically in the nasopharynx. However, in susceptible individuals, such as elderly and immunocompromised people and children, the bacteria may become pathogenic, adhere to nasopharyngeal tissue, and cause conditions such as community acquired pneumonia, meningitis, septicemia, acute sinusitis, otitis media, conjunctivitis, meningitis, bacteremia, sepsis, osteomyelitis, septic arthritis, endocarditis, peritonitis, pericarditis, cellulitis, and brain abscess.

According to methods of the disclosure, the methods also encompass inhibiting new, and compromising existing microbial adherence to pharyngeal surface tissue. As used herein, the term “inhibiting new and compromising existing microbial adherence” may be used to describe releasing bacteria adhered to pharyngeal tissue, weakening biofilm resistance to shear force by reducing bacterial adhesion to biofilm, damaging bacterial capsule or cell wall, promoting bacterial cell edema, preventing metabolism of sucralose due to adhesin conformational changes, and cell lysis. Alternatively, inhibiting microbial adherence may prevent bacteria from adhering to pharyngeal tissue through 95% or more reduction in bacterial carriage and the concomitant elimination of biofilm that results with bacterial reduction at the 95% or more level.

Inhibiting new and compromising existing microbial adherence may facilitate elimination of bacteria that cannot adhere to pharyngeal tissue from pharyngeal environments. For instance, inhibiting microbial adherence using a method of the present disclosure may eliminate about 20, 30, 40, 50, 60, 70, 80, 90, or 100% of bacteria that may adhere to pharyngeal surface tissue from pharyngeal environments. Preferably, inhibiting new and compromising existing microbial adherence using a method of the present disclosure eliminates about 80, 85, 90, 95, or about 100%, more preferably about 90, 91, 92, 93, 94, 95, 96, 97, 98, 99, or about 100% of bacteria that may adhere to and colonize pharyngeal surface tissue from pharyngeal environments.

By inhibiting adherence of microorganisms to pharyngeal surface tissue, a method of the present disclosure may reduce swelling and inflammation of pharyngeal tissue, and treat any condition that may be caused by attachment of pathogenic microorganisms to pharyngeal surface tissue. Additionally, in the oropharynx, an environment starved of sucrose may also reduce production of lactic acid by Lactobacilli, thereby raising the pH in the oral cavity, inhibiting the growth of acid-producing bacteria, and favoring remineralization of tooth enamel. Further, an increase in remineralization of tooth enamel affords the ability to tolerate a lower salivary pH without caries formation. Thus, the methods and compositions described herein also prevent caries formation. (C. Dawes, J. Can. Dent. Assoc. 69:11, 722, (December 2003)). As used herein, “treating a pharyngeal condition” refers to preventing the development of a pharyngeal condition, preventing the progression of a clinical condition, and reducing the severity of a clinical condition.

A pharyngeal condition may be an oropharyngeal condition, a nasopharyngeal condition, or a laryngopharyngeal condition. Non-limiting examples of pharyngeal conditions that may be treated using a method of the present disclosure include gingivitis, periodontal disease, dental caries, halitosis, community acquired pneumonia, pharyngitis, meningitis, septicemia, acute sinusitis, otitis media, conjunctivitis, bacteremia, sepsis, osteomyelitis, chronic rhinitis, septic arthritis, endocarditis, peritonitis, pericarditis, cellulitis, dermatitis, tonsillitis, and brain abscess. Preferably, a method of the present disclosure is used to treat a condition selected from otitis media, gingivitis, dental caries, halitosis, dermatitis, sinusitis including fungal sinusitis, pharyngitis, chronic rhinitis, tonsillitis, and sepsis, more preferably, gingivitis, dental caries, halitosis, acute sinusitis, and otitis media.

It should be noted that the methods described herein are not limited to treatment of conditions of oral cavity. In non-limiting example, although other bacteria such as S. Mutans, lactobacillus, and other acidophilic bacteria are not present in the upper respiratory tract, pneumococcal bacteria are known to survive in the nasopharanx through adhesion to, and a parasitic relationship with the host. For example, S. pneumoniae cleaves sialic acid from human glycoconjugates to be used as a carbohydrate source. Although sucrose is not generally present the ear canals, Eustachian tubes, or sinuses, pneumococcal bacteria do metabolize glucose from the host to produce polysaccharides that comprise biofilm. Thus, a sucralose composition as described herein is effective in treating conditions of the upper respiratory tract, while a composition of a sugar alcohol, such as xylitol, is not. Sucralose degrades biofilm by inhibiting adhesion of bacteria to the film. Xylitol will work only if bacteria ingest it.

Other microbial conditions that can be treated with the compositions and according to the methods described herein include skin conditions such as skin ulcers. Further, because microbes including bacteria and fungi spread to the eye from the sinuses through the tear ducts, the present disclosure contemplates ophthalmic solutions for treatment of inflamed and itchy eyes.

Thus, for use according to the present disclosure, the pharyngeal surface tissue or other tissue contacted with the composition comprising an antimicrobial effective concentration of sucralose. When a pharyngeal surface tissue is nasopharyngeal tissue, surface tissue is contacted with a composition comprising an antimicrobial effective concentration of sucralose using a spray, nasal drops, or a mist. Preferably, nasopharyngeal surface tissue is contacted with a composition comprising an antimicrobial effective concentration of sucralose using nasal spray. When a pharyngeal surface tissue is oropharyngeal surface tissue, surface tissue is contacted with a composition comprising an antimicrobial effective concentration of sucralose using a spray, drops, or a mist. Preferably, oropharyngeal surface tissue is contacted with a composition comprising an antimicrobial effective concentration of sucralose using drops.

A dose of a composition of the present disclosure that may be administered using a nasal spray, drops, nebulizer, lavage or other delivery method can and will vary depending on the subject, the condition being treated, the concentration of sucralose in the composition, and the pharyngeal surface tissue being administered. A dose of a composition is generally sufficient to coat the surface of the intended pharyngeal tissue. The dose of a spray may be controlled by varying the volume of each administered puff from a spray bottle, by administering multiple puffs, or a combination thereof. For instance, 1, 2, 3, 4, or 5 puffs of a composition of the present disclosure may be administered. Preferably, 1, 2, or 3 puffs of a composition of the present disclosure are administered. The dose of drops may be controlled by varying the number of administered drops. For instance, 1, 2, 3, 4, or 5 drops of a composition of the present disclosure may be administered. Preferably, 2, 3, or 4 drops of a composition of the present disclosure are administered. The volume of a drop or a puff of a composition of the present disclosure may be about 1 μl to about 500 μl or more.

Administering multiple in vivo doses of a composition of the present disclosure per day to a sucrose-free or substantially sucrose-free treatment area may also be used as needed to provide the desired level of treatment. Administration of multiple daily doses may maintain a pharyngeal environment starved of sucrose for a longer period of time, and may improve treatment of pharyngeal conditions. The elimination of 95% or more of pathogenic bacteria raises pH to around 6 or 7. At this pH level, conditions are deleterious to acidophilic bacteria. Other bacterial species achieve and can maintain dominance in and on epithelium tissue. Dominance of non-acidophilic bacteria eliminates disease and renders daily consumption of sucrose harmless to the host's epithelium.

For instance, 1, 2, 3, 4, or more doses of the composition may be administered per day. Preferably, 1, 2, 3, 4, 5, 6, 7, 8, 9, 10, or more doses of the composition are administered per day. More preferably, 2, 3, 4, 5, 6, or 7 doses of the composition are administered per day. Additionally, it is preferred that a composition is administered daily to maintain a pharyngeal environment starved of sucrose and treat pharyngeal conditions.

### C. Kits

In yet another aspect, the present disclosure provides a kit for treating a pharyngeal condition in a subject. A kit comprises any composition described herein, and instructions for administering said composition or following any of the methods described herein. The composition comprises for example an antimicrobial effective concentration of sucralose, wherein the antimicrobial effective concentration of sucralose is at least about 1% w/w or more, and preferably at least about 1% w/w up to about 25% w/w. Compositions may be as described in Section I, and methods of using such compositions may be as described in Section II above.

Compositions of the present disclosure are preferably packaged in any container capable of delivering drops, a spray, a mist, a foam, a wash, a gel, a paste or a coating. An exemplary container is a container equipped with a spray device, such as a nasal or oral spray pump. Another exemplary container is a container equipped with a dropper. Alternatively, compositions of the present disclosure are preferably packaged in containers capable of delivering drops and a spray. For instance, such containers are capable of delivering a spray when held upright, and drops when held upside down.

A kit may also comprise two containers: a first container having a composition formulated for topical administration as a nasal spray, wherein the first container is equipped with a nasal spray pump; and a second container having a composition formulated for topical administration as dental drops, wherein the second container is equipped with a dropper.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure, therefore all matter set forth is to be interpreted as illustrative and not in a limiting sense.

### Example 1

### Preparation of Antimicrobial Sucralose Composition

Antimicrobial sucralose solution was prepared as follows: dry ingredients Xylitol, Hydroxyl Apatite, preservatives, and sodium chloride in the amounts listed below were placed in a dispensing bottle. Pure sucralose was added to the mixture. Optional fluoride solution was added. Water was added to fill the dispensing bottle. The resulting solution was mixed.

Sucralose source: Tate and Lyle (www.tateandlyle.com), pure sucralose only, in liquid or solid form, between 1 and 25 parts w/w.
Xylitol between 1 and 25 parts w/w
Fluoride—1000 ppm
Hydroxyl Apatite—5 nanomoles/Litre Preservative (Phenyl-carbinol, benzalkonium chloride—5%-10% w/w)

### Example 2

### Maintenance of Pharyngeal Health

A dropper bottle and a nasal spray bottle were used to deliver to subjects a composition comprising about 1% to about 5% w/w sucralose as described in Example 1. The composition was administered to the nasopharynx 3-5 times daily using the spray bottle. Two puffs were used for each administration. The composition was also administered to the mouth using the dropper bottle. The dropper bottle was used to deliver 2-3 drops at bed time after brushing teeth.

Healthy gums, teeth and sinuses of the subject were maintained throughout the period of use of the sucralose composition.

### Example 3

### Shear Force Adhesion Test I

Saliva from a volunteer was cultured in a culture tube with 15 ml of TY media contained a glass slide until. The tube was incubated at 37 degrees C. for 3 days. 1 ml of 20% sucrose was added to fresh media daily after aseptic transfer to a sterile container. After 3 days, the sample separated into two culture test tubes, a positive control and a product sample. Addition of one milliliter of 20% sucrose solution to the positive control for one day maintained bacterial growth.

The product sample contained one milliliter of dental drops solution added to the sample on day 3 at the same time that aseptic transfer to a new culture test tube occurred. Sucrose was not added to the TY culture media since sucrose metabolism is a competitive process. After addition of dental drops product to the culture broth, the sample incubated for 24 hours at 37 degrees Celsius.

Sufficient growth was present on both glass slides after 24 hours of incubation to allow staining and microscopic visualization. Slides from TY media broth were stained with crystal violet (5 mg/ml in 2% ethanol), then placed on a slide holder for a shear force assay test. The assay measured the amount of biofilm removed by shear force due to applied fluid flow.

Shear force created by application of a fluid stream, specifically Milli-Q water, was the result of a pressure head applied at an angle of 10.5 degrees. Fluid flow rate was 11.3 ml/s through a ¼ inch diameter orifice. Flow rate was maintained constant through use of an Erlenmeyer flask reservoir and ¼inch tubing

The pressure head was 1852 N/m2. The slide normal force was 1.61 N. The tangential force was 0.3 N. Flow to the slide was 5 seconds in duration. This shear force was 100 times greater than shear force in experiments of bacterial growth in shear flow (Stoodley, 2000). The Reynolds number for flow in the tube is 3408 indicating that flow in the tube is transitory. Flow at the tube orifice was turbulent flow. After application of shear force, the slides were kept moist, but excess water was removed. Biofilm remained on both slides. It was determined that sufficient biofilm was present for before and after optical comparison

Slides were stained prior to shear force application. Images were compared for the before and after application of shear force samples. This comparison indicated inferior adhesion for the product sample compared to the control.

Both control and product samples contained complete biofilm coverage of the slide surface prior to application of shear force. Shear force application began at top of the slide near the slide center. Water flowed over the rest of the slide. Optical image locations were near the slide center.

Presence of hydroxyapatite (HA) crystals is expected as the product sample contained HA in solution. Some calcium and phosphate precipitated out of the TY media solution forming HA crystals on the slide's surface.

Biofilm is present on both the control and product slides prior to application of shear force. After application of shear force, the control slide remains coated with bacterial biofilm (bacteria and extra-cellular polysaccharides.

### Example 4

### Culture Media Exposure Experiment Part I

A culture sample from two volunteers mixed with a dose of dental drops and imaged for an hour after mixing is in FIGS. 5 and 6.

Whole saliva cultured in TY media and incubated at 37 degrees Celsius. Hemacytometry and pH measurements were made daily.

The control sample had no exposure to Dental Drops (DD) until the product mixed with culture media in vitro on a glass slide.

The product sample was whole saliva from a volunteer with long-term DD product use in vivo. The product sample mixed with DD in vitro on a glass slide.

### Example 5

### Culture Media Exposure Experiment Part II

The image in FIG. 7 below is of a product use sample prior to exposure to DD product on a glass slide.

FIG. 8 show a culture sample from a product use volunteer mixed with a dose of dental drops and imaged after mixing (FIG. 8). The product sample was whole saliva from a volunteer with three or more years of consistent DD product use.

FIG. 9 shows a sample from a volunteer with no product use 40 minutes after sample exposure to DD by mixing on a glass slide. The sample was whole saliva from the volunteer. Protocols in use were from the NIH's “Protocols to study the physiology of oral biofilms”.

### Example 6

### Shear Force Assay Test II Part 1: In Vivo Product Use

Shear force assay Optical Microscopy (OM) data was obtained for biofilms grown over an eleven-day period. OM data is from slides cultured for a volunteer user of Dental Drops (DD) labeled “In vivo product use”, and a volunteer who does not use dental drops labeled “No in vivo product use”. All culture preparation and maintenance was aseptic and in a hood. Glass slides were inside a sealed culture tube with 30 ml of TY media. Whole saliva from volunteers inoculated the TY media. Culture and maintenance was in accordance with the NIH “Protocols for testing the physiology of oral biofilms” document (http://www.ncbi.nlm.nih.gov/pmc/articles/PMC3130507/). Daily cell count and pH readings monitored the cultures. Shear force assay tests for samples with in vivo exposure only were run after 9 days incubation and biofilm growth. Shear force assays for samples with product exposure were exposed to 1% product solution for 22 hours prior to testing.

There were three samples for both volunteers. The samples are:

1. X—Biofilm exposed to DD

2. C—Biofilm not exposed to any product

3. L—Biofilm exposed to Listerine antiseptic.

The test matrix is set forth in Table 1.

TABLE 1
Shear force assay test matrix

| | Control-C | DD product-X | Listerine-L |
|---|---|---|---|
| Product | No product | DD product | Antiseptic product |
| Volunteer | exposure | exposure | exposure |
| Control | No product | DD product | Antiseptic product |
| Volunteer | exposure | exposure | exposure |

On day 9, the shear force test began with removal of the control samples from media, methylene blue stain, and examination under a Leica DMI4000 microscope. In order to eliminate differences between individuals, OM data was obtained for both volunteer samples before shear force was applied, and after to qualitatively determine the amount of biofilm removed by shear force. A separate adhesion assay provides quantitative data for biofilm adhesion.

Differences will result between in vivo product use and no in vivo product use (control) samples. Long-term use of dental drops reduces adhesion of biofilm. There may be changes to bacterial colonies or individual bacteria with long-term use of the product. In Table 1, “in vivo product use” refers to exposure in vivo for three or more years. Note that any difference is due to exposure that occurred ten days or more prior to the shear force test. No product exposure occurred during the 10-day incubation period. During this 10-day period, no product was added to the cultures, but the cultures were provided with 30 ml of fresh TY media aseptically every 24 or 48 hours.

Published results for shear force measurements on biofilm range from the most basic, a stream of tap water, to sophisticated test over many days in a centrifuge test apparatus [(Stoodley, 2000)]. Shear force to the slides was from MQ water passed from a water reservoir through a tube of inner diameter 0.25 inch placed 4.5 inches above the elevated end of the inclined slide. The slide was inclined at an angle of 10.5 degrees. Flow rate of the water was 11.4 ml/s on average +/−0.05 ml/s. Slide position was under the stream of water from the tube at the high end of the slide. Flow duration was 5 seconds. Shear force was 0.3 N. Flow outside the tube was turbulent flow with Reynolds number 3408.

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

What is claimed is:

1. A method of treating a pharyngeal condition in a subject, the method comprising delivering to a pharyngeal surface tissue in the oral cavity of the subject a composition comprising an antimicrobial effective concentration of sucralose, wherein the antimicrobial effective concentration of sucralose is at least about 1% w/w.

2. The method of claim 1, wherein the composition is sucrose-free.

3. The method of claim 1, wherein the oral cavity of the subject is substantially sucrose-free when the composition is delivered.

4. The method of claim 1, wherein delivery of the composition is at least once daily over a period of at least about 1 week.

5. The method of claim 1, wherein delivery of the composition is at least once daily over a period of at least about 1 month.

6. The method of claim 1, further comprising maintaining the oral cavity substantially sucrose-free during a post-administration period immediately following delivery of the composition to a pharyngeal surface tissue.

7. The method of claim 6, wherein the post-administration period is at least about 5 hours.

8. The method of claim 6, wherein the post-administration period is about 6-8 hours.

9. The method of claim 6, wherein the post-administration period is during a daily period of sleep.

10. The method of claim 6, wherein maintaining the post-administration period substantially sucrose-free comprises avoiding food and drink during the post-administration period.

11. The method of claim 1, wherein the administration to the subject comprises contacting the pharyngeal surface tissue with the composition.

12. The method of claim 1, wherein a pharyngeal condition is selected from the group of swelling and inflammation of pharyngeal tissue, otitis media, gingivitis, periodontal disease, dental caries, halitosis, dermatitis, sinusitis, pharyngitis, chronic rhinitis, tonsillitis, cough and chest congestion.

13. The method of claim 1, wherein the pharyngeal surface tissue is nasopharyngeal surface tissue and the nasopharyngeal surface tissue is contacted with the composition using a nasal spray formulation.

14. The method of claim 12, wherein the nasal spray formulation is administered 3 to 5 times daily with 2 puffs per nostril per administration.

15. The method of claim 1, wherein the pharyngeal surface tissue is oropharyngeal surface tissue and the composition is administered topically using a dental drop formulation.

16. The method of claim 14, wherein 2 to 3 drops of the dental drop formulation is administered during and after tooth brushing.

17. An antimicrobial composition, wherein the composition comprises an antimicrobial effective concentration of sucralose, wherein the antimicrobial effective concentration of sucralose is at least about 1% w/w or more.

18. The antimicrobial composition of claim 16, wherein the composition is substantially sucrose-free.

19. The antimicrobial composition of claim 16, wherein the composition is a sucralose solution comprising about 1% to about 25% w/w sucralose.

20. The antimicrobial composition of claim 18, wherein the sucralose solution is a buffered saline solution.

21. The antimicrobial composition of claim 19, wherein the buffered saline solution comprises about 0.35-0.45% w/w sodium chloride and about 0.1% to about 0.2% w/w hydrogen carbonate.

22. The antimicrobial composition of claim 18, wherein the sucralose solution further comprises a preservative.

23. The antimicrobial composition of claim 21, wherein the preservative is benzalkonium chloride.

24. The antimicrobial composition of claim 18, wherein the composition is formulated for topical administration as a nasal spray.

25. The antimicrobial composition of claim 18, wherein the composition is formulated for topical administration as dental drops.

26. The antimicrobial composition of claim 18, further comprising at least one additive selected from the group consisting of a desensitizing agent, a flavoring agent, a coloring, a fluoride source, an emulsifier, a remineralizing agent, an antimicrobial agent, an anticalculus agent, an antioxidant, a vitamin, a teeth whitening agent, and a combination of any thereof.

27. The antimicrobial composition of claim 18, further comprising hydroxylapatite or calcium phosphate.

28. A kit comprising at least one container containing an antimicrobial composition and instructions for administering said composition, wherein said composition comprises an antimicrobial effective concentration of sucralose, wherein the antimicrobial effective concentration of sucralose is at least about 1% w/w or more.

29. The kit according to claim 27, wherein the composition is substantially sucrose-free.

30. The kit according to claim 27, wherein the composition is a sucralose solution comprising about 1% to about 25% w/w sucralose.

31. The kit according to claim 27, wherein the sucralose solution is a buffered saline solution.

32. The kit according to claim 30, wherein the buffered saline solution comprises about 0.35-0.45% sodium chloride and about 0.1% to about 0.2% hydrogen carbonate.

33. The kit according to claim 27, wherein the sucralose solution further comprises a preservative.

34. The kit according to claim 32, wherein the preservative is benzalkonium chloride.

35. The kit according to claim 27, wherein the composition is formulated for topical administration as a nasal spray, and the container is a spray bottle.

36. The kit according to claim 27, wherein the composition is formulated for topical administration as dental drops, and the container is a dropper bottle.

37. The kit according to claim 27, wherein the kit comprises a first container having a composition formulated for topical administration as a nasal spray, wherein the first container is a spray bottle, and a second container having a composition formulated for topical administration as dental drops, wherein the first container is a dropper bottle.

38. The kit according to claim 27, wherein the kit comprises a container having a composition formulated for topical administration, wherein the container is a bottle capable of delivering the composition as a nasal spray and as dental drops.

39. The kit according to claim 27, wherein the composition further comprises at least one additive selected from the group consisting of a desensitizing agent, a flavoring agent, a coloring, a fluoride source, an emulsifier, a remineralizing agent, an antimicrobial agent, an anticalculus agent, an antioxidant, a vitamin, a teeth whitening agent, a breath freshening agent, and a combination of any thereof.

40. The kit according to claim 27, wherein the composition further comprises hydroxylapatite or calcium phosphate.
